(19) United States
(12) Patent Application Publication
(10) Pub. No.: US 2009/0123043 A1
(43) Pub. Date: May 14, 2009

(54) Method and System for Correlating Image and Tissue Characteristic Data

(76) Inventors: Kazuhiro Gono, Sagamihara-shi (JP); Takeshi Suga, Tokyo (JP)

Correspondence Address: GIBBONS P.C., ONE GATEWAY CENTER, NEWARK, NJ, 07102, US

(21) Appl. No.: 11/937,153

(22) Filed: Nov. 8, 2007

(51) Int. Cl. A61B 5/00

(52) U.S. Cl. 382/128

(57) ABSTRACT

Capsule type endoscopes generate large amounts of in-vivo image data that requires review and analysis by a doctor or clinician. By not reviewing the images gathered from healthy tissue, and only focusing on images indicating potential abnormalities, the time it takes to review the data can be greatly reduced. By correlating the tissue images with a characteristic known to indicate a potential abnormality, only the suspect images need to be reviewed.

### CROSS REFERENCE TO RELATED APPLICATIONS

This application is related to U.S. patent application Ser. No. ______, filed concurrently herewith, entitled “Capsule Blood Detection System and Method” and U.S. patent application Ser. No. ______, filed concurrently herewith, entitled “Blood Content Detecting Capsule,” both of which are herein incorporated by reference.

### FIELD OF THE INVENTION

This invention relates to a system and method for analyzing and reviewing large amounts of diagnostic data. More specifically, the invention is directed to a system and method for reviewing large amounts of image data and blood content data collected from an in-vivo detection system.

### BACKGROUND OF THE INVENTION

The use of capsule-type endoscopes has become more widely used in the field of medicine. A capsule-type endoscope typically contains an imaging device such as a camera or CCD device and traverses the digestive tract of a patient. Because of the extensive path taken by a capsule-type endoscope, large amounts of data and images are generated.

Recently, it has been discovered that certain light scattering and absorption techniques may be utilized to detect abnormal living tissue by detecting an early increase in microvascular blood supply. Such applications known as “Early Increase in Blood Supply” have been found to assist with in vivo tumor imaging, screening, and detecting. EIBS may reveal in tissues that are close to, but are not themselves, affected, precursors to lesion or tumor that precede the development of such lesions or tumors. The technique for utilizing EIBS as an early detection method has been disclosed in the article entitled Increased Microvascular Blood Content is an Early Event in Colon Carcinogenesis, Wali et al., Gut April 2005; 54: 654-660, which is incorporated herein by reference. A technique for detecting Hb concentration using polarized light has been disclosed in Y. L. Kim, Y. Liu, R. K. Wali, H. K. Roy, M. J. Goldberg, A. K. Kromin, K. Chen, and V. Backman, Simultaneous measurement of angular and spectral properties of light scattering for characterization of tissue microarchiftecture and its alteration in early precancer, IEEE J. Sel. Top. Quant. Elec., Vol. 9, 243256 (2003) and M. P. Siegel, Y. L. Kim, H. K. Roy, R. K. Wali, and V. Backman, Assessment of blood supply in superficial tissue by polarization-gated elastic light-scattering spectroscopy, Applied Optics, Vol. 45, 335-342 (2006) and the entirety of those articles are incorporated herein by reference.

There are numerous techniques known for detecting abnormality in tissues, and most if not all require human analysis. For example, to utilize all the data collected from a capsule-type endoscope as a diagnostic tool, the large number of images must be inspected frame by frame and analyzed by a doctor or clinician to diagnose whether there are any abnormalities present in the patient. In some instances, because of the large amount of images captured, it can take several hours to review the data, only to determine that there are no abnormalities present.

Traditionally, to review such data, images were displayed on a screen, and indicator or cursor was moved manually by the user in a sequential fashion from one image to the next. This required the user to view all the gathered images without any prescreening of the images to determine if certain areas are of higher importance or of a particular interest. Accordingly, the present invention provides a advantageous techniques for assisting in the screening and analysis of data to aid in the detection of abnormal tissue using EIBS and optical measurements.

### BRIEF SUMMARY OF THE INVENTION

One aspect of the invention is directed to a method for screening data from a capsule endoscope includes capturing images of living tissue from a body lumen, detecting a first characteristic of the living tissue in the area of the tissue of the captured images, and correlating the captured images with the respective data values indicative of the characteristic. More specifically, the invention is directed toward a method of searching large amounts of captured images by focusing a doctor's or clinician's attention to those images correlated with respective detected tissue characteristics that meet specific criteria. By utilizing such a method, the time a doctor or clinician has to spend analyzing normal data is greatly reduced. One such way to practice this invention involves the correlation of tissue image data captured from a capsule-type endoscope with a tissue characteristic of the imaged tissues, such as blood content data, collected from the same capsule. By synchronizing the data based on time or some other criteria, a doctor or clinician can review the images in the areas of abnormal blood content data and bypass normal healthy areas, thereby reducing the number of images that need to be reviewed.

Another aspect of the invention, discloses a system for screening blood content data and image data collected from a capsule endoscope where the capsule contains a blood content detector and an image capture device such as a camera or CCD for capturing images from a patient. The system also includes a processor to process the blood content data and captured image data collected by the capsule. In this aspect of the invention, the system would also include a display to allow a doctor or clinician to view the data or representations of the data.

In still another aspect of the invention, it is contemplated that the system provides a visual indication on the display of the captured images that correspond to the areas of abnormal blood content values. By displaying the correlated images, a doctor or clinician may evaluate the blood content data and the image data together to determine what course of treatment is necessary for a patient.

In another aspect of the invention, the system automatically presents the user with selected sections of the collected correlated data meeting a predetermined condition. A feature of the present invention allows the user to view areas of interest in a rapid fashion, thereby reducing the number of images that the user is reviewing.

### BRIEF DESCRIPTION OF THE DRAWINGS

For a better understanding of the present invention, reference is made to the following description and accompanying drawings, while the scope of the invention is set forth in the appended claims:

FIG. 1 shows a block diagram of an exemplary capsule-type endoscope in accordance with the invention.

FIG. 2 shows a block diagram of an exemplary system utilizing a capsule-type endoscope device in accordance with the invention.

FIG. 3 shows a flow diagram employed in an exemplary system practicing the invention.

FIG. 4 shows representative correspondence between captured image data and correlated characteristic data.

FIG. 5 depicts an exemplary embodiment of the present invention.

### DETAILED DESCRIPTION OF THE INVENTION

The present invention concerns a system and method for correlating large amounts of captured images collected from tissue with values indicative of a detected characteristic taken proximate the imaged tissue. More specifically, the invention concerns a system and method for correlating detected blood content data and the corresponding tissue image data to improve the analysis process.

Referring to the drawings, like numbers indicate like parts throughout the views as used in the description herein, the meaning of “a “an,” and “the” includes plural reference unless the context clearly dictates otherwise. Also, as used in the description herein, the meaning of “in” includes both “in” and “on” unless the context clearly dictates otherwise. Also, as used in the description herein, the meanings of “and” and “or” include both the conjunctive and disjunctive and may be used interchangeably unless the context clearly dictates otherwise.

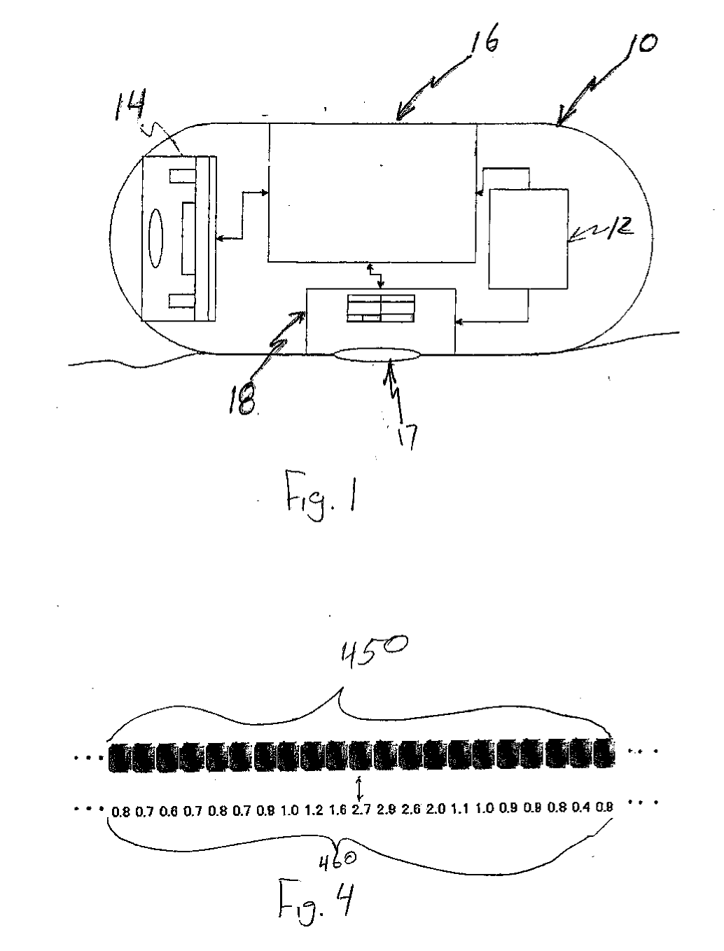

FIG. 1 depicts a block drawing of an exemplary capsule type endoscope usable in accordance with the invention. Capsule 10 houses power supply 12, imaging unit 14, transmitter 16, capsule window 17, and blood content detector 18.

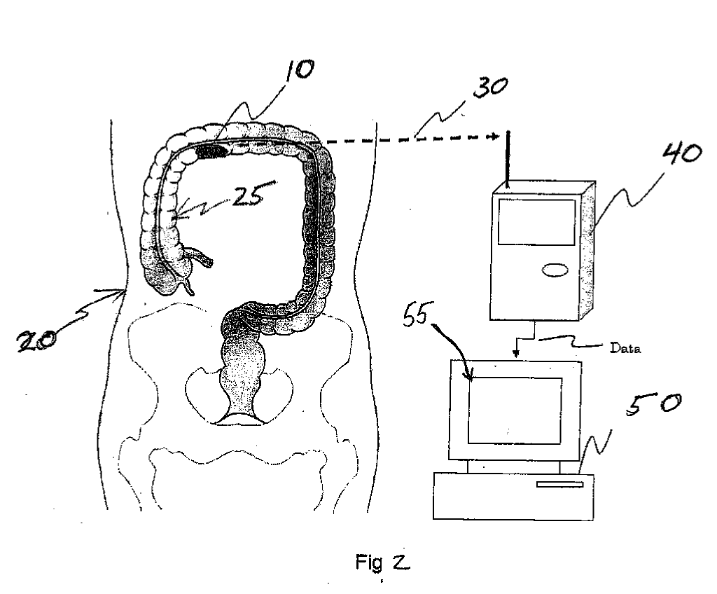

FIG. 2 shows the representative components of the data screening system of the present invention utilizing a capsule type endoscope. It will be appreciated, however, that the system described is not limited to capsule endoscopes but encompasses the use of traditional endoscopes as well. Referring to FIGS. 1 and 2, capsule endoscope 10 is swallowed by patient 20 and travels through the patient's digestive tract 25. Tissue image data and blood content data collected by imaging unit 14 and blood content detector 18 are transmitted via transmitter 16 as signal 30 to receiving unit 40. Receiving unit 40 may contain a processor for processing image data and blood content data, and may also include a display unit 55. Alternatively, receiving unit 40 may act merely as a data receiver to receive the signal 30 and convey the information to image processing unit 50. Image processing unit 50 may be any type of general or special purpose computer or processor capable or receiving, processing, and displaying the received data. Processing unit 50 may even be a server with access to the Internet, thereby allowing a remote user or clinician to analyze the captured data over the Internet.

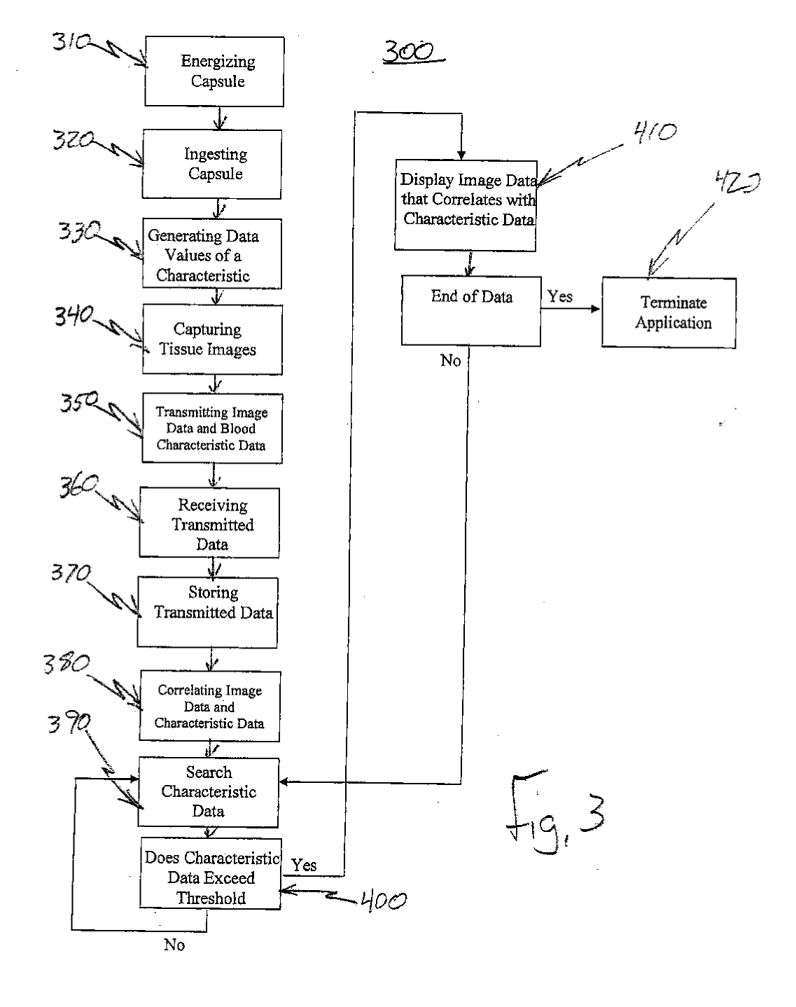

FIG. 3 shows a flow diagram 300 of an exemplary method of the present invention. The flow diagram 300 will be described with reference to the capsule and system of FIGS. 1 and 2. In step 310, capsule endoscope 10 is energized or activated. Such activation is not critical to practicing the invention and can be accomplished by a variant of ways including techniques known in the art. Such techniques include the use of an on-board battery, induction, RF excitation, or the like. In step 320, Capsule 10 is ingested by patient 20 and traverses the patient digestive tract 25. A detector in capsule 10, such as blood content detector 18 generates data values throughout the transit of the digestive tract 25. The data generated in step 330 may be related to any characteristic that may be collected from the digestive tract. Such data characteristics may include for example, blood content data, pH data, temperature data, or any other data that might be utilized in aiding diagnosis or prediction of any abnormal condition or characteristic.

Then, in step 340, imaging unit 14 captures images of the tissue proximately surrounding the site from which the characteristic data of step 330 is generated. Both captured image data and characteristic data are transmitted in step 350 from capsule endoscope 10 via transmitter 16 to receiving unit 40. The particular method chosen for transmission of signal 30 is not critical for the invention and may be by any well know method such as an RF transmission. Receiving unit 40 receives signal 30 and either stores the received data according to steps 360 and 370 or provides the data to processing unit 50. Receiving unit 40 may be part of processing unit 50 or may be a standalone unit. Alternatively, processing unit 50 may contain receiving unit 40 in a single integrated device. In accordance with step 380, the characteristic data gathered in step 330 and the tissue images gathered in step 340 are correlated based on a common attribute. It should be noted that correlating step 380 may also be carried out in the capsule prior to transmission of data to receiving unit 40. This may be performed by a processor associated with the blood content detector 18 or imaging unit 14 or a separate processor not depicted in FIG. 1. Typically, the attribute of correlation is time based; however, other attributes may be used.

A user such as a doctor or clinician may then interact with processing unit 50 in accordance with step 390 to search the characteristic data for data that meets specific criteria as identified by step 400. When blood content data is the characteristic data that is collected in step 330, a threshold level, or other suitable criteria such as range, minimum/maximum, or statistical analysis, is typical used to determine if the blood content data is within a normal range. If the characteristic data being analyzed in step 400 meets a preset threshold or other criteria, the tissue image data that correlates to that characteristic data is displayed to the user thereby allowing the user to review the surrounding tissue in the area proximate to the suspect characteristic data.

Once the user analyzes that particular characteristic data and correlated image, the process continues and steps 390 to 410 are repeated as long as there is data to analyze. Once the user has reviewed all characteristic data that meets the data threshold criteria, the process is complete. It will be appreciated that by utilizing this method of scanning the correlated characteristic data for areas of data that meet a preset criteria, and only reviewing images where there is an indication of a higher probability of abnormal results, will greatly reduce the time it takes a doctor or clinician to review the data collected from a patient. Furthermore, it will be appreciated that this method is not limited to use in a capsule type endoscope, but can be employed by any number of image gathering techniques including traditional endoscopes fitted with characteristic data detectors, such as blood content detectors and an imaging device.

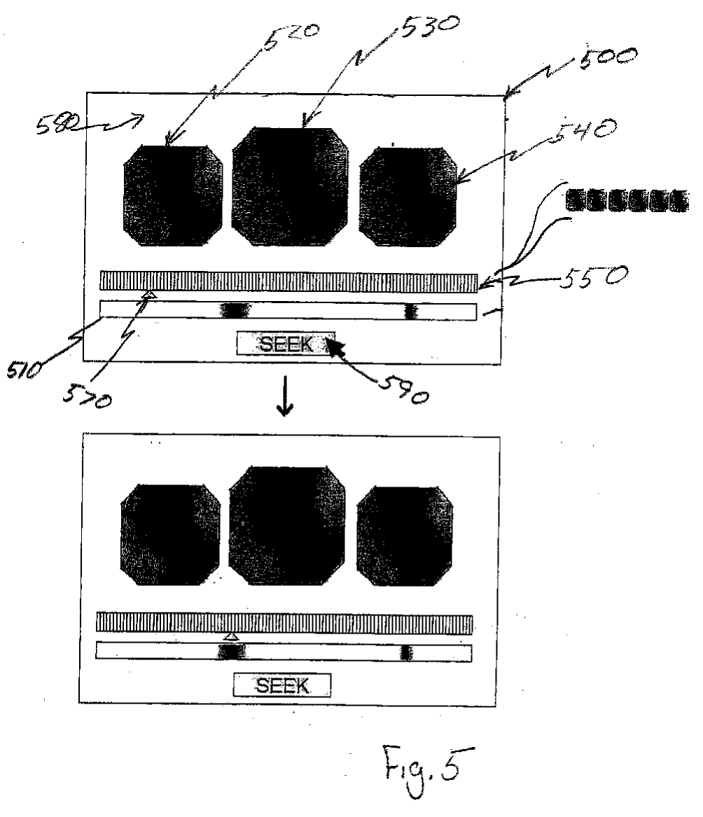

FIG. 4, shows the correlated relationship between characteristic data 460 captured in step 330 and image data 450 captured in step 340. As can be seen in FIG. 4, the characteristic data can be stored and or displayed as a simple numerical value and quickly searched to find areas that meet specific criteria. Once the user or system locates the characteristic data 460, that meets the criteria, the image data 450 is quickly accessible to the viewer, because of the ability to correlate the data.

FIG. 5 represents an exemplary embodiment of the present invention. Display screen 500 may be incorporated into receiving unit 40 or processing unit 50 and is intended to allow the user to view the images captured in step 340 of FIG. 3. Areas of reduced hemoglobin concentration captured by a blood content detector or the like, are represented in grey scale in display area 510. Index area 530 contains thumbnail images of the tissue images captures in step 330 of FIG. 3. Indicator 570 represents the area being analyzed by the user. Image 520 represent the image directly preceding the image of interest 530 and image 540 represents the image directly after the image of interest 530.

A selectable on-screen icon or button 590 allows the user to automatically jump through the data focusing only on the areas of interest. By selecting button 590, the data is automatically scrolled to the next area of interest, thereby automatically bypassing normal data that does not indicate any abnormalities. By selecting button 590, data of interest is also displayed in display area 580. Images 520 and 540 represent the images directly before and directly after the image correlated to the blood content data that displays low hemoglobin characteristics, as exemplified in steps 400 and 410 of FIG. 3. By repeatedly selecting or activating button 590, the images continue to jump to the next location at which the blood content data displays low hemoglobin content. By utilizing a desired algorithm, it is possible to selectively review a sequence of images in which an area of abnormal tissue may have been imaged among a large number of sequenced images. As a result, the data is analyzed in an efficient and highly accurate manner. As will be appreciated, other implementations of utilizing correlated characteristic data with gathered images may be employed without departing from the present invention.

What is claimed is:

1. A method for screening data from a capsule endoscope comprising the steps of:
capturing images of living tissue from a body lumen,
generating data values based on detecting a first characteristic of the living tissue proximate said tissue in said captured images, and
correlating the respective data values with the respective captured images.

2. The method of claim 1 further comprising the steps of:
identifying areas of interest based on the characteristic data values, and
displaying at least one captured image and data value for the identified areas of interest.

3. The method of claim 1 wherein the first characteristic data is blood content.

4. The method of claim 1 wherein the characteristic data and corresponding captured images are synchronized in time.

5. A system for screening blood content data and image data collected from a capsule endoscope comprising:
a capsule comprising a blood content detector for detecting blood content in living tissue and an image capture device for capturing images of living tissue,
a processor for processing blood content and image data from said capsule, and
a display for displaying the results from the processor.

6. The system of claim 5 wherein the processor provides an indication on the display for those captured images that correspond to blood content values that satisfy a condition.

7. The system of claim 6 wherein said display enables a user to directly access selected images that correspond to blood content values that satisfy the condition.

8. The system of claim 6 wherein the condition is a threshold value.

9. The system of claim 6 wherein the condition is a range of values.

10. The system of claim 5 wherein the processor correlates blood content data and the captured image data, and wherein the display displays the captured image data based on a characteristic of the correlated blood content data.
